(19) United States
(12) Patent Application Publication
(10) Pub. No.: US 2015/0218647 A1
(43) Pub. Date: Aug. 6, 2015

(54) BIOMARKERS FOR IDENTIFYING ESOPHAGEAL CANCER PATIENTS FOR TREATMENT WITH AN ANTI-EGFR DRUG

(71) Applicant: CROWN BIOSCIECNCE, INC., Santa Clara, CA (US)

(72) Inventors: Henry Qixiang Li, Carlsbad, CA (US); Jie Yang, Beijing (CN); Jianyun Deng, Beijing (CN)

(21) Appl. No.: 14/418,693

(22) PCT Filed: Jul. 31, 2013
(86) PCT No.: PCT/US13/53092

§ 371 Date: Jan. 30, 2015

(30) Foreign Application Priority Data
Jul. 31, 2013 (CN) PCT/CN2013/053092

(51) Int. Cl. C12Q 1/68; A61K 45/06; A61K 39/395; C07K 16/28; C07K 16/40

(52) U.S. Cl. CPC C12Q 1/6886; C07K 16/2863; C07K 16/40; A61K 39/39558; A61K 45/06; C12Q 2600/158; C12Q 2600/106; C07K 2317/24; C07K 2317/76

(57) ABSTRACT

The present invention provides methods for the treatment of esophageal cancer patients as well as the identification and selection of esophageal cancer patients for treatment with an anti-EGFR agent treatment including anti-EGFR antibody treatment, e.g., cetuximab treatment. In some embodiments, the drug is against a heterodimer formed by EGFR and another member of the ErbB receptor family such as EfbB2/Her2/neu.

### CROSS-REFERENCE TO RELATED APPLICATIONS

The present application claims priority to International Patent Application No. PCT/CN2012/079411, filed on Jul. 31, 2012, which is incorporated herein by reference in its entirety.

### FIELD OF THE INVENTION

The present invention relates to treatment of esophageal cancer patients as well as identification and selection of esophageal cancer patients for treatment with a drug against epidermal growth factor receptor (EGFR), such as an anti-EGFR antibody treatment, e.g., cetuximab.

### BACKGROUND

Esophageal carcinoma (ESC) is one of the deadliest cancers with a 5 year survival of less than 10% (The National Oesophago-Gastric Cancer Audit: An audit of the care received by people with Oesophago-gastric Cancer in England and Wales. Third Annual Report London, NHS Information Centre; 2010). There are two major histology types of ESC: squamous cell carcinoma (SCC) and adenocarcinoma (ADC). Besides surgery, the standard treatment options are chemoradiotherapy (CRT). All of these have limited effect. Esophageal SCC is particularly prevalent in part of China, and there is no effective target therapy. There remains a need in the art for esophageal carcinoma therapies.

The present invention meets this need and provides methods for the effective treatment of esophageal carcinoma.

### SUMMARY OF THE INVENTION

The present invention provides methods for treating esophageal carcinoma in a patient by an anti-epidermal growth factor receptor (EGFR) agent.

In some embodiments, the methods comprise administering to the patient an effective amount of an anti-EGFR agent. In some embodiments, the drug is directly targeting EGFR. In some embodiments, the drug is targeting the signaling pathway downstream of EGFR. In some embodiments, the drug is an antagonist or an antibody of the ligand of EGFR, for example, an antagonist or an antibody of epidermal growth factor (EGF), transforming growth factor α (TGFα), HB-EGF, amphiregulin, betacellulin, epigen, and/or epiregulin. In some embodiments, the drug is a small molecule. In some embodiments, the drug is against a heterodimer formed by EGFR and another member of the ErbB receptor family such as EfbB2/Her2/neu. In some embodiments, the drug is against a homodimer formed by EGFRs. In some embodiments, the drug is an anti-EGFR agent including anti-EGFR antibody treatment, e.g., cetuximab.

The present invention further provides for methods of treatment with an anti-EGFR agent, wherein the patient being treated with an anti-EGFR agent has one or more EGFR biomarkers. In some embodiments, the anti-EGFR agent comprises anti-EGFR antibody. In some embodiments, the anti-EGFR antibody comprises Cetuximab.

The present invention also provides for a detecting the presence or absence of one or more EGFR biomarkers and treating a patient with anti-EGFR agent when one or more EGFR biomarkers are present. In some embodiments, the anti-EGFR agent comprises anti-EGFR antibody. In some embodiments, the anti-EGFR antibody comprises Cetuximab.

The present invention further provides methods for identifying responder and/or nonresponder patients to an anti-EGFR agent treatment. In some embodiments, the anti-EGFR agent comprises anti-EGFR antibody. In some embodiments, the anti-EGFR antibody comprises Cetuximab. Responder and nonresponder patients are identified by detecting the presence or absence of one or more EGFR biomarkers in a biological sample that is obtained from a patient with esophageal carcinoma. According to the present methods, the presence of one or more EGFR biomarkers is indicative of a responder to an anti-EGFR agent treatment and the absence of one or more EGFR biomarkers is indicative of a nonresponder to an anti-EGFR agent treatment.

The present invention also provides methods for determining the treatment regimen for treating esophageal carcinoma in a patient. In this aspect, the method includes detecting the presence or absence of one or more EGFR biomarkers in a biological sample from a patient with esophageal carcinoma. The presence of one or more EGFR biomarkers indicates that the patient is a responder to an anti-EGFR agent treatment and the absence of an EGFR biomarker indicates that the patient is a nonresponder to an anti-EGFR agent treatment. In some embodiments, the anti-EGFR agent comprises anti-EGFR antibody. In some embodiments, the anti-EGFR antibody comprises Cetuximab. The patient is then treated with an anti-EGFR agent treatment when one or more EGFR biomarkers are present.

The present invention also provides methods for altering or modifying the treatment regimen of an anti-EGFR agent treatment by detecting the presence or absence of one or more EGFR biomarkers in a biological sample from a patient receiving the standard of care of esophageal carcinoma or an anti-EGFR agent treatment and altering or modifying the treatment regimen based on the presence or absence of one or more EGFR biomarkers in said biological sample. In some embodiments, the anti-EGFR agent comprises anti-EGFR antibody. In some embodiments, the anti-EGFR antibody comprises Cetuximab. For example, when one or more EGFR biomarkers are present an anti-EGFR agent treatment is continued and when one or more EGFR biomarkers are absent an anti-EGFR agent treatment is discontinued.

The present invention further provides methods of selecting patients with esophageal carcinoma for treatment with an anti-EGFR agent treatment. These methods include detecting the presence or absence of one or more EGFR biomarkers in a biological sample from said patient, wherein the presence of one or more EGFR biomarkers is indicative of a responder to an anti-EGFR agent treatment and the absence of an EGFR biomarker is indicative of a nonresponder to an anti-EGFR agent treatment. Patients with the presence of one or more EGFR biomarkers are then selected for treatment with an anti-EGFR agent treatment. In some embodiments, the anti-EGFR agent comprises anti-EGFR antibody. In some embodiments, the anti-EGFR antibody comprises Cetuximab.

For the methods of the present invention, the EGFR biomarker can be any in vitro or in vivo indicator for the activity of EGFR gene, and/or amplification, EGFR expression, EGFR RNA levels, EGFR activity, EGFR pathway activation or EGFR pathway signaling, all of which can be either increased or decreased as compared to a control group. In one embodiment, EGFR biomarkers include any form of mutations at the DNA, RNA, or protein level that are associated with EGFR activity, e.g., EGFR activation or gene amplification. In another embodiment, EGFR biomarkers include any measurement directly or indirectly associated with EGFR activity, e.g., EGFR activation or gene amplification. In yet another embodiment, EGFR biomarkers include L858R/T790M double mutations, insertion mutation (exon 20:2319-2320 AACCCCCAC), and deletion mutation (exon 19:2236-2350). In still another embodiment, EGFR biomarkers include any biomarker associated with EGFR activity and in the context of esophageal cancer with SCC histology. In some embodiments, the method comprises detecting the protein expression level of EGFR. The protein expression level of EGFR can be determined by any suitable methods known to one skilled in the art. In some embodiments, the protein expression level is determined by immunohistochemistry (IHC), western blot, protein immunestaining, protein immuneprecipitation, immuneeletrophoresis, immunoblotting, BCA assay, spectrophotometry, mass spectrometry or enzyme assay.

The present invention also provides methods for providing useful information for determining, evaluating or monitoring the treatment or the efficacy of treatment of esophageal carcinoma. The method includes determining the presence or absence of one or more EGFR biomarkers in a biological sample from a patient and providing the determination of the presence or absence of said one or more EGFR biomarkers to an entity that provides a determination or evaluation of the treatment or the efficacy of the treatment based on the presence or absence of one or more EGFR biomarkers.

The present invention also provides for kits. The kits include a reagent for measuring one or more EGFR biomarkers in a biological sample and optionally an instruction for using the one or more EGFR biomarker results for the determination of esophageal carcinoma treatment. For example, the presence of one or more EGFR biomarkers is indicative of a responder to an anti-EGFR agent treatment and the absence of one or more EGFR biomarkers is indicative of a nonresponder to an anti-EGFR agent treatment.

### BRIEF DESCRIPTION OF THE DRAWINGS

FIG. 1 shows representative ESC-SCC HuPrime® responses to cetuximab. Mice received 1 mg of cetuximab or vehicle control (saline) as shown.

FIG. 2 shows the pharmacodynamic effect of cetuximab in ESC-SCC models. Single dose treatment with the same agents as described in FIG. 1. The GA022 tumor samples were harvested at the time points as indicated for IHC analysis of biomarker pERK: IHC images (A) and IHC scores (B). Representative photos (400×) displayed the positive nuclear and cytoplasm staining of pERK biomarker in GAMO22 xenografts with single dose treatment. There was no detectable immunostaining when first antibody was replaced with normal rabbit IgG as a negative control.

FIG. 3 summarizes cetuximab activity and EGFR parameters. Panels from top to bottom show quantification of tumor response as measured by ΔT/ΔC values, EGFR mRNA expression, and EGFR expression as measured by gene chip analysis.

FIG. 4 shows a representative FISH analysis. Left panel: ES110 P5 and Right panel: patient sample (PA).

FIG. 5 shows bar graphs quantitating Panels from top to bottom show quantification of tumor response as measured by ΔT/ΔC values, EGFR expression as measured by gene chip analysis, EGFR gene copy number (PICNIC) and EGFR gene copy number (PennCNV).

FIG. 6 shows bar graph summary of results. Panels from top to bottom show quantification of tumor response as measured by ΔT/ΔC values, EGFR expression as measured by gene chip analysis, EGFR gene copy number (PICNIC) and EGFR gene copy number (PennCNV).

### DETAILED DESCRIPTION OF THE INVENTION

The present invention is based in part on the discovery that anti-EGFR agents can be used to treat esophageal cancer. In addition, it is also based in part on the discovery that EGFR biomarkers can be used as predictive biomarkers for determining whether a patient will respond to an anti-EGFR agent treatment. Accordingly, the present invention provides methods for treating esophageal cancer as well as methods for identifying esophageal cancer patients suitable for treatment with an anti-EGFR agent treatment. In some embodiments, the drug is targeting EGFR. In some embodiments, the drug is targeting the signaling pathway downstream of EGFR. In some embodiments, the drug is an antagonist or an antibody of the ligand of EGFR, for example, an antagonist or an antibody of epidermal growth factor (EGF), transforming growth factor α (TGFα), HB-EGF, amphiregulin, betacellulin, epigen, and/or epiregulin. In some embodiments, the drug is a small molecule. In some embodiments, the drug is against a heterodimer formed by EGFR and another member of the ErbB receptor family such as EfbB2/Her2/neu. In some embodiments, the drug is against a homodimer formed by EGFRs.

The present invention provides methods for treating esophageal carcinoma in a patient comprising administering to the patient an effective amount of an anti-EGFR agent treatment. In some embodiments, the patient being treated with an anti-EGFR agent has one or more EGFR biomarkers.

In some embodiments, the present invention provides methods for treating esophageal carcinoma in a patient with one or more EGFR biomarkers including administering to the patient an effective amount of an anti-EGFR agent treatment. In some other embodiments, the present invention provides methods for treating esophageal cancer in a patient with one or more EGFR biomarkers including administering to the patient an effective amount of an anti-EGFR antibody treatment, e.g., cetuximab.

In some embodiments, the present invention provides methods for treating esophageal cancer in a patient including detecting the presence or absence of one or more EGFR biomarkers. A patient is then treated with an effective amount of an anti-EGFR agent treatment when one or more EGFR biomarkers are present.

In some embodiments, the present invention also provides methods for identifying responder and nonresponder patients comprising detecting the presence or absence of one or more EGFR biomarkers in a biological sample from a patient with esophageal carcinoma. The presence of one or more EGFR biomarkers indicates a responder to an anti-EGFR agent treatment and the absence of one or more EGFR biomarkers indicates a nonresponder to an anti-EGFR agent treatment.

In some embodiments, the present invention provides methods for determining a treatment regimen for treating esophageal carcinoma in a patient. Such methods include detecting the presence or absence of one or more EGFR biomarkers in a biological sample from a patient with esophageal carcinoma. The presence one or more EGFR biomarkers is indicative of a responder to an anti-EGFR agent treatment and the absence of an EGFR biomarker is indicative of a nonresponder to an anti-EGFR agent treatment. The methods can further include treating a patient with an anti-EGFR agent treatment when one or more EGFR biomarkers are present.

In some embodiments, the present invention provides methods for altering the treatment regimen of an anti-EGFR agent treatment. These methods include detecting the presence or absence of one or more EGFR biomarkers in a biological sample and altering the treatment regimen based on the presence or absence of one or more EGFR biomarkers. When one or more EGFR biomarkers are present treatment with an anti-EGFR agent treatment is continued and in some cases altered according to methods known in the medical arts and when one or more EGFR biomarkers are absent treatment with an anti-EGFR agent treatment is discontinued.

In some embodiments, the present invention provides methods for selecting a patient with esophageal carcinoma for treatment with an anti-EGFR agent treatment comprising detecting the presence or absence of one or more EGFR biomarkers in a biological sample from a patient. The presence of one or more EGFR biomarkers is indicative of a responder to an anti-EGFR agent treatment and the absence of an EGFR biomarker is indicative of a nonresponder to an anti-EGFR agent treatment. The methods further provide for selecting for treatment with an anti-EGFR agent treatment those patients with the presence of one or more EGFR biomarkers.

The presence of one or more EGFR biomarkers is indicative of a responder to an anti-EGFR agent treatment. In some embodiments, the one or more EGFR biomarkers are examined prior to an anti-EGFR agent treatment. In some embodiments, the one or more EGFR biomarkers are examined during the anti-EGFR agent treatment. In some embodiments, the one or more EGFR biomarkers are examined after the anti-EGFR agent treatment.

A responder according to the present invention is an individual who exhibits treatment efficacy and a nonresponder does not exhibit treatment efficacy. The phrase “determining the treatment efficacy” or “determining the efficacy of treatment” and variants thereof can include any methods for determining that a treatment is providing a benefit to a subject. The term “treatment efficacy” and variants thereof are generally indicated by alleviation of one or more signs or symptoms associated with the disease and can be readily determined by one skilled in the art. “Treatment efficacy” may also refer to the prevention or amelioration of signs and symptoms of toxicities typically associated with standard or non-standard treatments of a disease, i.e. chemotherapy or radiation therapy for the treatment of cancer. Determination of treatment efficacy is usually indication and disease specific and can include any methods known or available in the art for determining that a treatment is providing a beneficial effect to a patient. For example, evidence of treatment efficacy can include but is not limited to remission of the disease or indication, for cancer this can include but is not limited to a decrease or reduction in tumor size, in tumor metastasis, etc. Further, treatment efficacy can also include general improvements in the overall health of the subject, such as but not limited to enhancement of patient life quality, increase in predicted subject survival rate, decrease in depression or decrease in rate of recurrence of the indication (increase in remission time). (See, e.g., Physicians' Desk Reference (2010).)

An anti-EGFR agent treatment can include any treatment containing one or more entities that can increase, decrease, eliminate, enhance, delay, reduce, or block the activity of EGFR signaling pathway. In some embodiments, the composition is directly against EGFR, or one or more components in EGFR signaling pathway, at DNA level, transcriptional level, translational level, post-translational level, and/or protein level. The composition can specifically target EGFR, or target at least EGFR. In some embodiments, the composition can cause gene suppression and/or gene silencing of EGFR and/or a component in EGFR signaling pathway, e.g., knocking down or knocking out EGFR and/or a component in EGFR signaling pathway. In some embodiments, the composition can modify EGFR protein activity, such as modifying the EGFR binding activity to its ligand and/or its ability to induce downstream signaling pathways. In some embodiments, the drug is an antagonist or an antibody of the ligand of EGFR, for example, an antagonist or an antibody of epidermal growth factor (EGF), transforming growth factor α (TGFα), HB-EGF, amphiregulin, betacellulin, epigen, and/or epiregulin. In some embodiments, the drug can target to EGFR and/or the ligand and block ligand-receptor binding. In some embodiments, the drug can cause confirmation changes in the receptor and/or the ligand and reducing or inactivating EGFR mediated cell signaling. In some embodiments, the drug is against a heterodimer formed by EGFR and another member of the ErbB receptor family such as EfbB2/Her2/neu, or a homodimmer formed by two EGFR molecules. EGFR signaling pathway is described in Sechacharyulu et al. (Targeting the EGFR signaling pathway in cancer therapy, Expert Opin Ther Targets, 2012 January; 16(1): 15-31.), Oda et al. (A comprehensive pathway map of epidermal growth factor receptor signaling, Molecular Systems Biology 1:2005.0010), and Development EGFR Signaling Pathway (Pathway Maps, Thomson Reuters, 2012), each of which is incorporated herein in its entirety for all purposes.

In some embodiments, the agent contains one or more entities inhibiting or decreasing the activity of EGFR, e.g., at DNA, RNA, or protein level. According to the present invention, an anti-EGFR agent treatment can include any anti-EGFR treatment containing one or more chemical compounds or compositions, biological molecules, or a combination thereof. In one embodiment, the anti-EGFR agent treatment of the present invention is an anti-EGFR antibody treatment. According to the present invention, an anti-EGFR antibody treatment can include any treatment using anti-EGFR antibody or antibody like therapeutics including without any limitation any molecule with one or more anti-EGFR CDRs. In one embodiment, anti-EGFR antibody treatment includes any approved anti-EGFR antibody, e.g., cetuximab (also known as erbitux) or biosimilar or derivatives thereof, e.g., fully human anti-EGFR antibody, etc. Cetuximab (marketed in North America by ImClone and Bristol-Myers Squibb and in the rest of the world by Merck KGaA) is a recombinant, human/mouse chimeric monoclonal antibody that blocks activation of the epidermal growth factor (EGF) receptor (EGFR). Cetuximab can be given by intravenous infusion for treatment of metastatic colorectal cancer and head and neck cancers. In some embodiments, cetuximab is formulated in a sterile colorless liquid of pH 7.0 to 7.4. In some embodiments, cetuximab is formulated at a concentration of 2 mg/mL in either 100 mg (50 mL) or 200 mg (100 mL). In some embodiments, cetuximab is formulated in single-use vials. In some embodiments, the cetuximab formulation includes 8.48 mg/mL sodium chloride, 1.88 mg/mL sodium phosphate dibasic heptahydrate, 0.41 mg/mL sodium phosphate monobasic monohydrate, and sterile water for injection. Methods and formulations for administering cetuximab are well known by those skilled in the medical art and any well known methods of administering cetuximab, dosing regimens for cetuximab or formulations for cetuximab are contemplated for use with the methods of the present invention. Detailed compositions and methods of using Cetuximab are described in U.S. Pat. Nos. 8,075,916, 7,977,336, 6,217,866, each of which is incorporated by reference in its entirety for all purposes.

In some embodiments, the anti-EGFR agent comprises a small molecule. As used herein, the term “small molecule” refers to a molecule having a molecular weight of less than 500 MW, wherein the drug is a non-peptidyl or peptide agent. In some embodiments, the drug comprises a protein or a polypeptide. In some embodiments, the drug comprises a hybrid molecule. In some embodiments, the drug is an antibody. In some embodiments, the drug is an anti-EGFR antibody. In some embodiments, the drug is an anti-EGFR ligand antibody. In some embodiments, the drug is a humanized anti-EGFR ligand antibody. In some embodiments, the antibody is a monoclonal antibody.

In some embodiments, the drug is an anti-EGFR antibody. In some embodiments, the drug is Cetuximab or functional variants or derivatives thereof. None limiting examples of anti-EGFR antibodies have been described in PCT publication Nos. WO/2011/140151, WO/2007/058823, WO/2011/080209, WO/2010/080463, WO/2012/020059, WO/2011/080209, WO/2011/059762, WO/2011/152525, WO/2011/140254, WO/2010/034441, WO/2011/156617, WO/2005/090407, WO/2013/006547, WO/2008/140493, WO/2011/156617, U.S. Pat. Nos. 5,942,602, 6,129,915, 7,723,484, 7,618,631, 7,598,350, and U.S. Patent Application Publication Nos. 20100166755, 20080274114, 20130142812, 20110158987, 20120107234, 20110117110, 20110287002, 20120149879, 20120282633, 20100009390, 20050238640, 20060154334, 20120231021 and 20130149299, each of which is incorporated herein by reference in its entirety for all purposes.

For the methods of the present invention, the EGFR biomarker can be any in vitro or in vivo indicator for EGFR gene amplification, EGFR expression, EGFR RNA levels, constitutively active EGFR, EGFR activity, EGFR pathway activation or EGFR pathway signaling, all of which either increased or decreased as compared to a control group. In one embodiment, EGFR biomarkers include any form of mutations at the DNA, RNA, or protein level that are associated with EGFR activity, e.g., EGFR activation or gene amplification. In another embodiment, EGFR biomarkers include any measurement directly or indirectly associated with EGFR activity, e.g., EGFR activation or gene amplification. In some embodiments, the EGFR biomarker is selected from EGFR gene amplification, increased EGFR expression, increased EGFR RNA levels, constitutively active EGFR and enhanced EGFR pathway activation or enhanced EGFR pathway signaling. In yet another embodiment, EGFR biomarkers include L858R/T790M double mutations, insertion mutation (exon 20:2319-2320 AACCCCCAC), deletion mutation (exon 19:2236-2350). In still another embodiment, EGFR biomarkers include any biomarker associated with EGFR activity and in the context of esophageal cancer with SCC histology.

In some embodiments the EGFR gene copy number is at least 3, 4, 5, 6, 7, 8, 9, 10 or more copies. In some embodiments, the increase is determined by comparing to one or more standard levels or by comparing to levels known in the art as standard levels. Methods of measuring gene amplification, increased expression, increased RNA or DNA levels, as well as determining whether a protein is constitutively active are well known in the art and any such methods can be employed with the present invention.

As used herein, the term “standard level” or a “reference level” refers to a standardized data or data set representing the average, representative features or characteristics of one or more biomarkers in a specific population of subjects. Such features or characteristics include, but are not limited to, transcript abundance, transcript stability, transcription rate, translation rate, post-translation modification, protein abundance, protein stability, and/or protein enzymatic activity, etc. In some embodiments, the specific population of subjects are consisting of about 5, about 10, about 20, about 50, about 100, about 200, about 300, about 400, about 500, about 1000, about 5000, about 10K, or more individual subjects. In some embodiments, all individual subjects are responsive to an anti-EGFR treatment. In some embodiments, all individual subjects are not responsive to an anti-EGFR treatment.

In some embodiments, the methods comprise comparing the EGFR biomarker profile of a patient to a standard level EGFR biomarker profile derived from a population of subjects responding to an anti-EGFR agent, wherein the subject is determined to be responsive to the anti-EGFR agent if the EGFR biomarker profile of the patient is within the standard level EGFR biomarker profile.

In some embodiments, the methods comprise comparing the EGFR biomarker profile of a patient to a standard level EGFR biomarker profile derived from a population of subjects not responding to an anti-EGFR agent, wherein the subject is determined to be not responsive to the anti-EGFR agent if the EGFR biomarker profile of the patient is within the standard level EGFR biomarker profile.

As used herein, the sentence “the EGFR biomarker profile of the patient is within the standard level EGFR biomarker profile” refers to that the EGFR biomarker profile been analyzed is similar to the predetermined EGFR biomarker profile, for example, the parameters describing the EGFR biomarker profile of the patient are close to the parameters describing the predetermined EGFR biomarker profile, or within the variation range of a predetermined EGFR biomarker profile, e.g., the parameters are within the variation range based on a confidence interval of 90% constructed from the parameters describing the predetermined EGFR biomarker profile.

The EGFR biomarker profile can be determined by any suitable methods known to one skilled in the art. In some embodiments, a biological sample is taken from a subject and analyzed. In some embodiments, the biological sample is then typically assayed from the presence of one or more gene expression products such as RNA, mRNA, cDNA, cRNA, protein, etc.

In some embodiments, mRNA from a biological sample is directly used in determining the levels of expression of one or more genes by hybridization. In some particular embodiments, RNA is obtained from a biological sample. The RNA is then transformed into cDNA (complementary DNA) copy using methods known in the art. In some particular embodiments, the cDNA is labeled with a fluorescent label or other detectable label. The cDNA is then hybridized to a substrate containing a plurality of probes of interest. A probe of interest typically hybridizes under stringent hybridization conditions to at least one DNA sequence of a gene signature. In certain embodiments, the plurality of probes are capable of hybridizing to the sequences derived from the gene biomarkers under the hybridization conditions. In some embodiments, the conditions comprise using 6×SSC (0.9 M NaCl, 0.09 M sodium citrate, pH 7.4) at 65° C. The probes may comprise nucleic acids. The term “nucleic acid” encompasses known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, peptide-nucleic acids (PNAs).

In certain cases, the probes will be from about 15 to about 50 base pairs or more in length. The amount of cDNA hybridization can be measured by assaying for the presence of the detectable label, such as a fluorophore. The quantification of the hybridization signal can be used to generate a score for a particular sequence or set of sequences in the gene signature for a particular patient or plurality of patients.

Included within the scope of the invention are DNA arrays or microarrays containing a plurality of sequences that hybridize under stringent hybridization conditions to one or more of the gene sequences of the biomarkers. An example of a substrate containing one or more probes of interest is a plurality of DNA probes that are affixed to a substrate. In certain embodiments, the substrate may comprise one or more materials such as gel, nitrocellulose, nylon, quartz, glass, metal, silica based materials, silica, resins, polymers, etc., or combinations thereof. Typically, the DNA probes comprise about 10-50 bp of contiguous DNA. In certain embodiments, the DNA probes are from about 20 to about 50 bp of contiguous DNA. In certain embodiments, the present invention relates to kits which comprising a microarray directions for its use. The kit may comprise a container which comprises one or more microarrays and directions for their use.

The biological sample may also be analyzed for gene expression of one or more gene biomarkers using methods that can detect nucleic acids including, but not limited to, PCR (polymerase chain reaction); RT-PCT (reverse transcriptase-polymerase chain reaction); quantitative or semi-quantitative PCR, etc. In certain embodiments, the levels of gene expression are measured by detecting the protein expression products of the genes or DNA sequences. The levels of protein products may be measured using methods known in the art including the use of antibodies which specifically bind to a particular protein. These antibodies, including polyclonal or monoclonal antibodies, may be produced using methods that are known in the art. These antibodies may also be coupled to a solid substrate to form an antibody chip or antibody microarray. Antibody or protein microarrays may be made using methods that are known in the art.

Any suitable methods of protein detection, quantization and comparison can be used, such as those described in Tschesche (Methods in Protein Biochemistry, ISBN Walter de Gruyter, 2011, ISBN 3110252368, 9783110252361), Goluch et al. (Chip-based detection of protein cancer markers, ProQuest, 2007, ISBN 0549463453, 9780549463450), Speicher (Proteome Analysis: Interpreting the Genome, Elsevier, 2004, ISBN 0080515304, 9780080515304), Albala et al. (Protein Arrays, Biochips and Proteomics, CRC Press, 2003, ISBN 0203911121, 9780203911129), Walker (The Protein Protocols Handbook, Springer, 2002, ISBN 0896039404, 9780896039407), Fung (Protein Arrays: Methods and Protocols, Springer, 2004, ISBN 1592597599, 9781592597598), and Bienvenut (Acceleration and Improvement of Protein Identification by Mass Spectrometry, Springer, 2005, ISBN 1402033184, 9781402033186), each of which is incorporated by reference in its entirety for all purposes. In some embodiments, the protein expression level of biomarkers are detected and measured by immunohistochemistry (IHC), western blot, protein immunestaining, protein immuneprecipitation, immuneeletrophoresis, immunoblotting, BCA assay, spectrophotometry, mass spectrometry or enzyme assay.

For additional methods related to detection, quantitation and comparison of biomarker levels, see, e.g., Current Protocols in Molecular Biology, Ed. Ausubel, Frederick M. (2010); Current Protocols in Protein Science Last, Ed. Coligan, John E., et al. (2010); Current Protocols in Nucleic Acid Chemistry, Ed. Egli, Martin (2010); Current Protocols in Bioinformatics, Ed. Baxevanis, Andreas D. (2010); and Molecular Cloning: A Laboratory Manual, Third Edition, Sambrook, Joseph (2001), all of which are incorporated herein by reference in their entirety.

Methods for obtaining biological samples are well known in the art and any standard methods for obtaining biological samples can be employed. Biological samples that find use with the methods of the present invention include but are not limited to serum, blood, plasma, whole blood and derivatives thereof, skin, hair, hair follicles, saliva, oral mucous, vaginal mucous, sweat, tears, epithelial tissues, urine, semen, seminal fluid, seminal plasma, prostatic fluid, pre-ejaculatory fluid (Cowper's fluid), excreta, biopsy, ascites, cerebrospinal fluid, lymph, and tissue extract sample or biopsy. (See, e.g., Clinical Proteomics: Methods and Protocols, Vol. 428 in Methods in Molecular Biology, Ed. Antonia Vlahou (2008).) In one embodiment, the biological sample of the present invention includes any cell or tissue samples of the esophagus, e.g., on site or circulating or migrating cells of esophageal cancer. In another embodiment, the biological sample of the present invention includes any extract or partial or whole fractionation of cell or tissue samples of the esophagus, e.g., on site or circulating or migrating cells of esophageal cancer.

In some embodiments, the patient suitable for treatment by the methods of the present invention is of Asian descent or African descent. In some embodiments the patient is of Asian descent and exhibits the presence of one or more EGFR biomarkers. In some embodiments, the patient is of East Asian descent.

In some embodiments, the esophageal carcinoma treated is esophageal squamous cell carcinoma (SCC). Esophageal cancers are typically carcinomas which arise from the epithelium, or surface lining, of the esophagus. Most esophageal cancers fall into one of two classes: squamous cell carcinomas, which are similar to head and neck cancer in their appearance and association with tobacco and alcohol consumption, and adenocarcinomas, which are often associated with a history of gastroesophageal reflux disease and Barrett's esophagus.

Any suitable test can be used to determine the histology of the cancer. Such test and examination include, but are not limited to, common signs and symptoms of esophaeal cancer, including but not limited to, backwards movement of food through the esophagus and possibly mouth (regurgitation), chest pain not related to eating, difficulty swallowing solids or liquids, heartburn, vomiting blood, hoarseness, chronic cough, hiccups, pneumonia, bone pain, bleeding into the esophagus, and weight loss, medical history and physical exam, imaging tests, chest X-ray, computed tomography (CT) scan, magnetic resonance imaging (MRI) scan, positron emission tomography (PET) scan, bone scan, sputum cytology, needle biopsy, bronchoscopy, endobronchial ultrasound, endoscopic esophageal ultrasound, mediastinoscopy and mediastinotomy, thoracentesis, thoracoscopy, immunohistochemistry, molecular tests, blood tests, barium swallow, endoscopic ultrasound, esophaogastroduodenoscopy (EGD) and biopsy, or any suitable methods derived from thereof.

In some embodiments, an anti-EGFR agent treatment can be co-administered with one or more chemotherapeutics, radiation therapeutics, chemoradiation therapeutics, or targeted therapeutics.

In some embodiments, the chemotherapeutic includes but is not limited to vinblastine, vincristine, dactinomycin, daunorubicin, doxorubicin, etoposide, mithramycin, paclitaxel, docetaxel, cisplatin, carboplatin, fluorouracil, folinic acid and irinotecan.

In some embodiments, the targeted therapeutic includes but is not limited to bevacizumab, trastuzumab, erlotinib, panitumumab, sorafenib, infliximab, adalimumab, basiliximab, daclizumab and omalizumab.

In some embodiments, the radiation therapeutic is administered at a dosage of about 40 Gy to about 80 Gy. In some embodiments the dosage is about 50 Gy to about 70 Gy, in some embodiments, the dosage is about 50 Gy to about 65 Gy. In some embodiments, the radiation therapy is administered at a dosage of about 50 Gy, about 55 Gy, about 60 Gy or about 65 Gy.

In some embodiments, the present invention provides methods for providing useful information for predicting, determining, evaluating or monitoring the treatment or efficacy of treatment of esophageal carcinoma with an anti-EGFR agent treatment. These methods include determining the presence or absence of one or more EGFR biomarkers in a biological sample from a patient and providing that information regarding the presence or absence of one or more EGFR biomarkers to an entity that provides a determination or evaluation of the treatment or efficacy based on the presence or absence of one or more EGFR biomarkers. If the one or more EGFR biomarkers are present, the entity can provide a determination that treatment with an anti-EGFR agent treatment should be used or should be continued. If the one or more EGFR biomarkers are absent, the entity can provide a determination that treatment with an anti-EGFR agent treatment should not be used or should be discontinued.

The present invention also provides for kits. The kits of the invention include at least one reagent for measuring one or more EGFR biomarkers in a biological sample and optionally an instruction for using the one or more EGFR biomarker results for the determination of esophageal carcinoma treatment. For example, the presence of one or more EGFR biomarkers indicates a responder to an anti-EGFR agent treatment and the absence of one or more EGFR biomarkers indicates a nonresponder an anti-EGFR agent treatment.

### EXAMPLES

### Example 1

### EGFR Gene Amplification Determines ESC Response to Cetuximab

### SUMMARY

Background: Esophageal squamous cell carcinoma (SCC) is a deadly malignance and particularly prevalent in part of China without option of effective target therapy. Cetuximab has been approved for treating EGFR-expressing metastatic colorectal carcinoma (mCRC) and head and neck squamous cell carcinoma, but yet to be tested for esophageal SCC.

Methods: We assessed antitumor activity of cetuximab in a cohort of naïve esophageal SCC patient derived xenograft (PDX) models, and identified predictive biomarkers that determine responders and non-responders by profiling these models for gene expression and oncogene mutations.

Findings: Among the 15 esophageal SCC PDX models tested, all responded to cetuximab to some degree, significantly higher response rates (RR) than those seen in other cancer types. The expression profiling and copy number variation analysis revealed that response is directly related to EGFR gene amplification and high expression. This observation suggests that EGFR is a key oncogenic driver for this disease. Cetuximab could be an effective treatment option for esophageal SCC patients and EGFR gene amplification/expression could be a predictive biomarker for this subset of ESC. Further prospective clinical investigation is warranted to support the expanding cetuximab indication into AC-SCC.

Esophageal carcinoma (ESC) is one of the deadliest cancers with a 5 year survival of less than 10% (The National Oesophago-Gastric Cancer Audit: An audit of the care received by people with Oesophago-gastric Cancer in England and Wales. Third Annual Report London, NHS Information Centre; 2010). There are two major histology types of ESC: squamous cell carcinoma (SCC) and adenocarcinoma (ADC). Besides surgery, the standard treatment options are chemoradiotherapy (CRT). All of these have limited effect. Esophageal SCC is particularly prevalent in part of China, and there is no effective target therapy.

Cetuximab is a monoclonal antibody that binds to EGFR and blocks its ligand induced downstream signaling. Cetuximab was approved by the Food and Drug Administration (FDA) for treating EGFR-expressing metastatic CRC (mCRC) without activating KRAS mutations at codons 12/13 (Ciardiello F and Tortora G. N Engl J Med. 2008 March 13; 358(11):1160-74), and also squamous cell carcinoma of head and neck (SCCHN) (Bonner J A, et al. Lancet Oncol. January; 11(1):21-8), a cancer type with resemblance to ESC-SCC. There are several phase II clinical trials on the combination treatments of cetuximab/chemotherapy agents for the advanced ESC. Few trials have been conducted to test cetuximab for activities in ESC patients (Okines A, et al. Nat RevClin Oncol. 2011 August; 8(8):492-503). In one trial, 57 ESC patients, 48 with ADC and 12 SCC, were treated in combination with CRT. 49/57 (70%) had a complete clinical response after CRT (Safran H, et al. Int J Radiat Oncol Biol Phys. 2008 February 1; 70(2):391-5). In a small study involving 5 gastric carcinoma (GC) and 5 ESC-SCC in China, better clinical benefit was seen for ESC-SCC, but the sample size was too small to make the conclusion (Qiu H-j, et al. Journal of Oncology. 2012; 16(4):294). SCOPE1, a 2-arm Phase II/III trial with confirmed ESC and testing definitive chemoradiotherapy with or without cetuximab, is currently ongoing in UK (Hurt C N, et al. BMC Cancer. 2011; 11:466). Cetuximab has yet to be tested for esophageal SCC.

Patient derived xenograft (PDX), without any in vitro manipulation, mirrors patients' histopathological and genetic profiles (Ding L, et al. Nature. 2010 April 15; 464(7291):999-1005; Marangoni E, et al. Clin Cancer Res. 2007 July 1; 13(13):3989-98; Nemati F, et al. Clin Cancer Res. 2010 April 15; 16(8):2352-62; Nemati F, et al. Anticancer Drugs. 2010 January; 21(1):25-32; Fichtner I, et al. Clin Cancer Res. 2008 October 15; 14(20):6456-68; and Hennessey P T, et al. PLoS One. 2011; 6(5):e20584). It has improved predictive power of preclinical cancer models and enables discovery of predictive biomarkers for targeted therapeutics. Due to the extensive diversity of cancer patient populations, successfulness of the clinical trial largely relies on the inclusion of the likely responders who express the intended target and have the right genetic profile, and the exclusion of non-responders. A large collection of PDX models can potentially reflect the diversity of tumors in patients, and thus can be used to test investigational targeted drugs by modeling clinical trial format.

A large collection of ESC PDX called ESC HuPrime® models was established. Antitumor activity of cetuximab was assessed in a cohort of 15 naïve esophageal SCC patient derived xenograft (PDX) models, and predictive biomarkers were also identified that determine responders and non-responders by profiling these models for gene expression and oncogene mutations. These data therefore suggest that cetuximab could be a new effective treatment option for patients of ESC-SCC, including specifically patients from East Asia.

### Materials and Methods

Patient tumor samples and engraftment in immunocompromised mice. The surgically removed fresh ESC-SCC tumor tissues were used to engraft into immunocompromised mice immediately after surgery. The subcutaneous engraftment has broadly been described by others (Marangoni E, et al. Clin Cancer Res. 2007 July 1; 13(13):3989-98). Briefly, the tumors were sliced into 3×3×3 mm³ fragments and inoculated subcutaneously on flanks of mice (Balb/c nude, 6-8 wks old, female, Beijing HFK Bioscience Co. Ltd., Beijing, China). The tumor growth was monitored twice a week. The established tumor models from these patient samples, called passage 0 or P0, were serially re-engrafted when tumors size reaches 500-700 mm³ (½ length×width²), called P1, 2, 3 . . . (<10) for passage and conduct studies (pharmacology, histopathology, immunohistology, cellular and molecular analysis). Access and use of the patient samples were approved by the Ethic Committee of Beijing Cancer Hospital along with the informed consents from patients. All procedures were performed under a sterilization conditions. All the experimental animals that involved in our studies were conducted in strict accordance with the recommendations in the Guide for the Care and Use of Laboratory Animals of the National Institutes of Health.

Evaluation of tumor response to cetuximab in PDX models. When tumor volume reaches 100-150 mm³, the mice were randomly grouped into two groups of 5 with similar average tumor volume. The first group was treated immediately after grouping with vehicle control (PBS, weekly IP injection for two weeks) and the second with cetuximab (weekly IP injection for two weeks, 1 mg/mouse, kindly provided by BMS). The tumor growth was monitored twice weekly, and % ΔT/ΔC value were calculate for assessing tumor response to cetuximab (ΔT=tumor volume change in the treatment group and ΔC=tumor volume change in control group).

Mutation analysis of model tumors. Oncogene hotspot analyses were carried out to identify the mutations in the tumors. Briefly, genomic DNA was extracted from the tissues using DNeasy mini kit (QIAGEN, Valencia, Calif.) per the manufacturer's instruction. All DNA samples will be PCR amplified in a 50 μL reaction. All detailed primer information is described in the supplement information. Polymerase chain reaction of 40 cycles was performed in 50 μL reaction mixtures containing: 100 ng of genomic DNA, 5 μL 10×PCR Buffer, 0.2 ρM each of primers, 0.2 mM 4×dNTPs and 1 μL TaqE. The amplified PCR products were gel purified and sequenced by Sanger Automated Sequencer (ABI). The data was analyzed using BioEdit software. For EGFR gene hotspot analyses, the primers were: EGFR-EXON19-F: 5-GTGCATCGCTGGTAACATCCA-3 (SEQ ID NO: 1); EGFR-EXON19-R: 5-GGAGATGAGCAGGGTCTAGAGCA-3 (SEQ ID NO: 2); EGFR-EXON20-F: 5-CGCATTCATGCGTCTTCACC-3 (SEQ ID NO: 3); EGFR-EXON20-R: 5-CTATCCCAGGAGCGCAGACC-3 (SEQ ID NO: 4); EGFR-EXON21-F: 5-TGGCATGAACATGACCCTGAA-3 (SEQ ID NO: 5): EGFR-EXON21-R: 5-CAGCCTGGTCCCTGGTGTC-3 (SEQ ID NO: 6). For KRAS hotspot analyses, the primers were: KRAS-EXON2-F: 5-TTATGTGTGACATGTTCTAAT-3 (SEQ ID NO: 7); KRAS-EXON2-R: 5-AGAATGGTCCTGCACCAGTAA-3 (SEQ ID NO: 8); KRAS-EXON3-F: 5-TCAAGTCCTTTGCCCATTTT-3 (SEQ ID NO: 9); KRAS-EXON3-R: 5-TGCATGGCATTAGCAAAGAC-3 (SEQ ID NO: 10); KRAS-EXON4-F: 5-TTGTGGACAGGTTTTGAAAGA-3 (SEQ ID NO: 11); KRAS-EXON4-R: 5-AGAAGCAATGCCCTCTCAAG-3 (SEQ ID NO: 12). PI3K-Exon 1, F: 5′-CTCCACGACCATCATCAGG-3′ (SEQ ID NO: 13) R: 5′-GATTACGAAGGTATTGGTTTAGACAG-3′ (SEQ ID NO: 14). PI3K-Exon 9, F: 5′-GATTGGTTCTTTCCTGTCTCTG-3′ (SEQ ID NO: 15), R: 5′-CCACAAATATCAATTTACAACCATTG-3′ (SEQ ID NO: 16), PI3K-Exon 20: F: 5′-TGGGGTAAAGGGAATCAAAAG-3′ (SEQ ID NO: 17), R: 5′-CCTATGCAATCGGTCTTTGC-3′ (SEQ ID NO: 18). c-MET-Exon 14, F: 5′-TGGGCACTGGGTCAAAGTCTC-3′ (SEQ ID NO: 19), R: 5′AACAATGTCACAACCCACTGAGGTA-3′ (SEQ ID NO: 20). c-MET-Exon 16, F: 5′-ATTAAATGTTACGCAGTGCTAAC-3′ (SEQ ID NO: 21), R: 5′-GGTTGCAAACCACAAAAGTAT-3′ (SEQ ID NO: 22). c-MET-Exon 17, F: 5′-GTATTCACTGTTCCATAATGAAGT-3′ (SEQ ID NO: 23), R: 5′GATGGCTGGCTTACAGCTAGTT-3′ (SEQ ID NO: 24). c-MET-Exon 18, F: 5′-AACAGTAGATGCTTAGTTTATGCT-3′ (SEQ ID NO: 25) R: 5′-AACAGATTC CTCCTTGTCACTT-3′ (SEQ ID NO: 26). c-MET-Exon 19, F: 5′-TTCTATTTCAGCCACG GGTAAT-3′ (SEQ ID NO: 27), R: 5′-ATGAAAGTAAAAGAGGAGAAACTC-3′ (SEQ ID NO: 28). c-MET-Exon 21, F: 5′-CACCCTAAAGCCGAAATGCG-3′ (SEQ ID NO: 29), R: 5′-CAAGGAGCAAAGAATATCGATGGC-3′ (SEQ ID NO: 30). AKT-Exon 3, F: 5′-ACATCTGTCCTGGCACAC-3′ (SEQ ID NO: 31), R: 5′-GCCAGTGCTTGTTGCTTG-3′ (SEQ ID NO: 32). BRAF-Exon 15, F: 5′-CTCTTCATAATGCTTGCTC-3′ (SEQ ID NO: 33), R: 5′-GTGAATACTGGGAACTATG-3′ (SEQ ID NO: 34). ERK-Exon 2, F: 5′-ACTTTACCAACT TGCCTTCT-3′ (SEQ ID NO: 35), R: 5′-TCACAACAAACCATCCCT-3′ (SEQ ID NO: 36). ERK-Exon 8, F: 5′-TGCCTTACCCATAAC-3′ (SEQ ID NO: 37), R: 5′-GGACCTTGAGGAACATAAT-3′ (SEQ ID NO: 38).

pERK-IHC and pEGFR-IHC staining of tumor slides. The standard immunohistochemistry (IHC) procedure was used in this study. Briefly, tumor tissues were fixed in 10% neutral buffered formalin and embedded in paraffin per standard histological procedures. After deparaffinization and rehydration, 3 μm thick tissue sections were pretreated at 95° C. in 0.01 M sodium citrate, pH 6.0 solution for 30 minutes, followed by staining with rabbit anti-human pERK (Cell Signaling, Boston, USA) or pEGFR (Epitomics, Burlingame, USA) antibodies. Positive staining was detected using Ultra Vision LP large Volume Detection System HRP Polymer (Ready-To-Use) Kit (Lab Vision, Fremont, Calif.). DAB was used as the chromogenic substrate, and sections were counterstained with Gill's Hematoxylin (Fisher Scientific, Fair Lawn, USA). The test specimens were then scored independently by three investigators in a blinded fashion according to the following criteria. Intensity Score: 0, no staining; 1+, minimal staining; 2+, moderate staining; 3+, strong staining. Areas of most intensity were identified by scanning tumor sections at low power (×100), and then images were photographed at high magnification (×400) using Olympus BX51 microscopy system with DP71 digital camera (Olympus, Melville, N.Y.).

CFISH analysis. FISH (dual-color) procedures were performed using Abbott PathVysion EGFR DNA Probe Kit per the manufacturer's protocol (Abbott, Downers Grove, Ill.). The Spectrum Orange fluorophore-labeled EGFR (303 kb) are specific for the EGFR gene locus on chromosome 7p12, and the Spectrum Green fluorophore-labeled chromosome enumerator probe (5.4 kb) targeted to the α-satellite DNA sequence located at the centromeric region of chromosome 7 (CEP7; 7p11.1-q11.1). Briefly, the FFPE sections were deparaffinized followed by digestion with pepsin and hybridization. The treated slides were denatured and hybridized with probes, followed by counterstaining with 15 μL DAPI/anti-fade solution and scanning using OLYMPUS BX51 fluorescent microscope (OLYMPUS BX51, Japan) equipped with single band pass filter set to detect DAPI, Rhodamine (7p12) and FITC (chromosome 7) at 1000×.

Expression profiling and gene copy number determination. Fresh HuPrime® tumor tissues were collected from the tumor-bearing mice, snap-frozen, and stored at −80° C. before being used for genetic and genomic analysis. For gene profiling analysis, the total RNA was isolated from the frozen tissues using Trizol (Invitrogen, Carlsbad, Calif.) per the manufacturer's instructions, and purified using RNeasy mini columns (Qiagen). RNA quality was assessed on a Bioanalyzer (Agilent). Only RNA samples with high quality (RI N>8) were used for expression profiling assays on Affymetrix HG-U219 array plates following standard protocol (GenChip® 3′IVT Express Kit User Manual, Affymetrix, P/N 702646 Rev. 8). Raw CEL data sets of all samples were normalized by RMA algorithm. Probe set intensity was expressed as Log(2) transformed values. For SNP/CNV assay using Affymetrix SNP6.0 chips, genomic DNA was isolated and purified using Genomic DNA Tissue and Blood Isolation Kit (Qiagen) by following manufacturer's instruction. DNA processing and chip hybridization were performed following standard Affymetrix protocol (Affymetrix® Genome-Wide Human SNP Nsp/Sty 6.0 User Guide, Affymetrix, P/N 702504, Rev 4). Raw CEL data was QC-ed and filtered to remove low call-rate samples, and gene copy number analysis was performed by PICNIC (Predicting Integral Copy Numbers In Cancer, see Greenman et al., PICNIC: an algorithm to predict absolute allelic copy number variation with microarray cancer data, Biostatistics, 11(1):164-175, 2010) and/or PennCNV methods, (Wang et al., PennCNV: an integrated hidden Markov model designed for high-resolution copy number variation detection in whole-genome SNP genotyping data Genome Research 17:1665-1674, 2007), each of the references is incorporated herein by reference in its entirety. For some of the samples, the relative gene copy numbers were determined by qPCR. Briefly, the same genomic DNAs were subjected to amplification using EGFR specific primers (EGFR-F: 5-CATGGTGAGGGCTGAGGTGA-3 (SEQ ID NO: 39); EGFR-R: 5-CCCCACCAGACCATGAGAGG-3 (SEQ ID NO: 40)) by SYBR Green based quantitative PCR. The mammalian LINE-1 retrotransposon gene was used as a reference. The q-PCR data was analyzed on the chromo4 system using Opticon Monitor 3 software to generate the raw data. The raw data was then processed using the delta CT relative quantification method. ACT=(CT value of target gene)-(CT value of reference gene). Delta CT values were then converted into intensity value (POWER (ΔCT,-2)). All data was normalized to that of a sample with known MET copy number to obtain relative MET copy number.

### Results

A large subset of ESC-SCC HuPrime® models responds to cetuximab. A large panel of esophageal carcinoma (ESC) HuPrime® models was tested by conducting a clinical trial-like study for assessing potential cetuximab activities against ESC. First, these models were established by transplanting the tumor tissues that were surgically removed from ESC patients into immunocompromised (Balb/c nude) mice by subcutaneous inoculation. The original patient diagnosis, along as model pathology confirmation, are shown in Table 1 and more details in Table 2. The majority of the established models are ESC-squamous cell carcinoma (ESC-SCC).

TABLE 1
Profiles of GC HuPrime ® model panels.

| | | | | EGFR | | | | | | | | | | |
|---|---|---|---|---|---|---|---|---|---|---|---|---|---|---|
| | | ΔT/ | Muta- | expres- | Gene | Gene | | | | | | | | |
| | | ΔC | tion | sion | CN | CN | k- | | | c- | | | | C- |
| Model | Histology | (%) | status | (Log2) | (PICNIC) | (PennCNV) | RAS | EGFR | PIK3CA | MET | AKT | BRAF | ERK | KIT |
| ESM219 | SCC | — | WT | 2.91 | — | 2 | WT | WT | WT | WT | WT | WT | WT | WT |
| ESM172 | SCC | — | WT | 3.76 | 5 | 2 | WT | WT | WT | WT | WT | WT | WT | WT |
| ESF212 | SCC | — | WT | 3.82 | 4 | 2 | WT | WT | WT | WT | WT | WT | WT | WT |
| ESM214 | SCC | — | WT | 4.08 | 5 | 2 | WT | WT | WT | WT | WT | WT | WT | WT |
| ESM215 | SCC | 44.2 | WT | 4.11 | 4 | 2 | WT | WT | WT | WT | WT | WT | WT | WT |
| ESF183 | | — | WT | 4.16 | 4 | — | WT | WT | WT | WT | WT | WT | WT | WT |
| ESM204 | SCC | 7.8 | WT | 5.11 | 5 | 2 | WT | WT | WT | WT | WT | WT | WT | WT |
| ESM136 | SCC | 38.3 | WT | 5.37 | 6 | 2 | WT | WT | WT | WT | WT | WT | WT | WT |
| ESM218 | SCC | — | WT | 5.38 | 5 | 2 | WT | WT | WT | WT | WT | WT | WT | WT |
| | with ADC | | | | | | | | | | | | | |
| ESM216 | Carcino- | — | WT | 5.83 | 5 | 2 | WT | WT | WT | WT | WT | WT | WT | WT |
| | sarcoma | | | | | | | | | | | | | |
| | or SCC | | | | | | | | | | | | | |
| ESF190 | SCC | — | WT | 5.84 | 5 | 2 | WT | WT | WT | WT | WT | WT | WT | WT |
| ESF148 | SCC | — | WT | 5.89 | 6 | — | WT | WT | WT | WT | WT | WT | WT | WT |
| ESF184 | SCC | | WT | 5.98 | — | 2 | WT | WT | WT | WT | WT | WT | WT | WT |
| ESM159 | SCC | — | WT | 6.09 | 5 | 2 | WT | WT | WT | WT | WT | WT | WT | WT |
| ESF147 | SCC | — | WT | 6.18 | 5 | 2 | WT | WT | WT | WT | WT | WT | WT | WT |
| ESM176 | SCC | — | WT | 6.23 | 4 | 2 | WT | WT | WT | WT | WT | WT | WT | WT |
| ESM199 | SCC | −4.0 | WT | 6.29 | 5 | 2 | WT | WT | WT | WT | WT | WT | WT | WT |
| ESM141 | SCC | — | WT | 6.31 | 5 | 2 | WT | WT | WT | WT | WT | WT | WT | WT |
| ESF630 | SCC | — | WT | 6.34 | 5 | 2 | WT | WT | WT | WT | WT | WT | WT | WT |
| ESM201 | SCC | — | WT | 6.37 | 6 | 3 | WT | WT | WT | WT | WT | WT | WT | WT |
| ESM026 | SCC | 23.2 | WT | 6.77 | 5 | — | WT | WT | WT | WT | WT | WT | WT | WT |
| ESF195 | SCC | −28.7 | WT | 6.83 | 6 | 3 | WT | WT | WT | WT | WT | WT | WT | WT |
| ESM042 | | −14.7 | WT | 7.82 | 6 | 3 | | | | | | | | |
| ESM178 | SCC | 0.10 | WT | 8.28 | 12 | 4 | WT | WT | WT | WT | WT | WT | WT | WT |
| ESF189 | SCC | — | WT | 9.39 | 15 | 4 | WT | WT | WT | WT | WT | WT | WT | WT |
| ESF191 | SCC | — | WT | 9.50 | 15 | 4 | WT | WT | WT | WT | WT | WT | WT | WT |
| ESM110 | | −33.4 | WT | 10.18 | 15 | 3 | | | | | | | | |
| ESM2116 | | — | — | — | — | — | | | | | | | | |

TABLE 2
ESC-SCC patient diagnosis and pathology and model pathology confirmation.

| ID | Histology | Gender | Age | Stage | Grade | Pathology Diagnosis |
|---|---|---|---|---|---|---|
| ESM219 | SCC | M | 53 | T3N0M0 II | II-III | moderately-poorly differentiated squamous |
| | | | | | | cell carcinoma of middle thoracic esophagus |
| | | | | | | (ulcerative); Squamous cell carcinoma from |
| | | | | | | middle thoracic part of esophagus, ulcerative |
| | | | | | | type, grade II-III (poorly differentiated), tumor |
| | | | | | | mass 4 cm × 3 cm × 1 cm, infiltrating |
| ESM172 | SCC | M | NA | T2N1M0 II B | II-III | squamous cell carcinoma of middle thoracic |
| | | | | | | esophagus (medullary); Squamous cell |
| | | | | | | carcinoma from middle thoracic part of |
| | | | | | | esophagus, medullary type, grade II-III |
| | | | | | | (moderately and poorly differentiated) tumor |
| | | | | | | mass 5 cm × 4 cm × 1 cm, infiltrating into deep |
| | | | | | | muscular |
| ESF212 | SCC | F | 58 | T1N1M0, II B | II-III | Squamous cell carcinoma from middle |
| | | | | | | thoracic part of esophagus, erosive type, grade |
| | | | | | | II-III (moderately-poorly differentiated), tumor |
| | | | | | | mass 2.5 cm × 2 cm × 0.3 cm, inf |
| ESM214 | SCC | M | 54 | T3N1M0 III | III | poorly differentiated squamous cell carcinoma |
| | | | | | | of upper thoracic esophagus; Squamous cell |
| | | | | | | carcinoma from superior thoracic part of |
| | | | | | | esophagus, submucosa nodular type, tumor |
| | | | | | | mass 3 cm × 2.5 cm × 1.5 cm, infiltrating into |
| | | | | | | fibrous membranes and serious atypical hyp |
| ESM215 | SCC | M | 74 | T1N0M0 I | II | carcinosarcoma (moderately differentiated |
| | | | | | | squamous cell carcinoma and liomyosarcoma) |
| | | | | | | of upper thoracic esophagus; Carcinosarcoma |
| | | | | | | by morphous from superior thoracic part of |
| | | | | | | esophagus (squamous cell carcinoma grade II |
| | | | | | | with leiomayosarcoma), intraluminal typ |
| ESF183 | | F | NA | NA | NA | NA |
| ESM204 | SCC | M | 76 | T2N0M0 II A | III | poorly differentiated squamous cell carcinoma |
| | | | | | | of esophagus (medullary); Squamous cell |
| | | | | | | carcinoma from inferior thoracic part of |
| | | | | | | esophagus, medullary type and poorly |
| | | | | | | differentiated, tumor mass |
| | | | | | | 5.5 cm × 4.5 cm × 1.5 cm, infiltrating into deep |
| | | | | | | muscular layer, no vis |
| ESM136 | SCC | M | 71 | T3N1M0 III | III | poorly differentiated squamous cell carcinoma |
| | | | | | | of middle thoracic esophagus (ulcerative); |
| | | | | | | Squamous cell carcinoma from middle thoracic |
| | | | | | | part of esophagus, ulcerative type, poorly |
| | | | | | | differentiated, tumor mass |
| | | | | | | 5.5 cm × 6 cm × 1.5 cm, infiltrating into fibrous |
| | | | | | | membrane |
| ESM218 | SCC | F | 49 | T3N0M0 II A | III | poorly differentiated squamous cell carcinoma |
| | | | | | | of middle thoracic esophagus with adeno- |
| | | | | | | differentiation; Squamous cell carcinoma with |
| | | | | | | adenoid differentiated from middle thoracic |
| | | | | | | part of esophagus, ulcerative type, grade III |
| | | | | | | (poorly differentiated), tumor ma |
| ESM216 | Carcinosarcoma | M | 54 | T1N0M0 | II/I | carcinosarcoma (moderately differentiated |
| | | | | | | squamous cell carcinoma and poorly |
| | | | | | | differentiated sarcoma) of middle |
| ESF190 | SCC | F | 66 | T3N0M0, II | II | squamous cell carcinoma of esophagus |
| | | | | | | (medullary) |
| ESF148 | SCC | F | 61 | T2N1M0 II B | III | squamous cell carcinoma of middle thoracic |
| | | | | | | esophagus (medullary) with adeno- |
| | | | | | | differentiation |
| ESF184 | SCC | F | 66 | T3N0M0 II A | I-II | Squamous cell carcinoma from middle |
| | | | | | | thoracic part of esophagus, medullary type, |
| | | | | | | grade I-II (well and moderately differentiated), |
| | | | | | | tumor mass 3 cm × 2.5 cm × 1 cm, infiltrating into |
| | | | | | | superficial musc |
| ESM159 | SCC | M | 63 | T3N0M0, II A | II | moderately differentiated squamous cell |
| | | | | | | carcinoma of esophagus ((annular |
| | | | | | | constricting); Squamous cell carcinoma from |
| | | | | | | superior thoracic part of esophagus |
| | | | | | | (postradiotherapy), scirrhous type, grade II |
| | | | | | | (moderately differentiated), tumor mass: |
| | | | | | | 3.5 cm × 1.5 cm × 0.5 c |
| ESF147 | SCC | F | 53 | T3N0M0 II A | III | squamous cell carcinoma of lower thoracic |
| | | | | | | esophagus (medullary); Squamous cell |
| | | | | | | carcinoma from inferior thoracic part of |
| | | | | | | esophagus, medullary type, grade III (poorly |
| | | | | | | differentiated), tumor mass 5 cm × 4 cm × 1.5 cm, |
| | | | | | | infiltrating into fibrous membranes with multi |
| ESM176 | SCC | M | 58 | T3N1M0 III | II | moderately differentiated squamous cell |
| | | | | | | carcinoma of middle thoracic esophagus |
| | | | | | | (medullary); Squamous cell carcinoma from |
| | | | | | | middle thoracic part of esophagus, medullary |
| | | | | | | type, grade II (moderately differentiated), |
| | | | | | | tumor mass 5 cm × 3.5 cm × 1 cm, infiltrating into f |
| ESM199 | SCC | M | 59 | T3N0M0 II A | III | squamous cell carcinoma of middle thoracic |
| | | | | | | esophagus (medullary); Squamous cell |
| | | | | | | carcinoma from middle thoracic part of |
| | | | | | | esophagus, medullary type, grade III (poorly |
| | | | | | | differentiated), tumor mass 4.5 cm × 4 cm × 1 cm, |
| | | | | | | infiltrating into fibrous membranes with gently |
| ESM141 | SCC | M | 64 | T2N1M0 II b | II | squamous cell carcinoma of middle thoracic |
| | | | | | | esophagus (ulcerative); Squamous cell |
| | | | | | | carcinoma from middle thoracic part of |
| | | | | | | esophagus, ulcerative type, grade II |
| | | | | | | (moderately differentiated), tumor mass |
| | | | | | | 7 cm × 2.5 cm × 1.5 cm, infiltrating into deep |
| | | | | | | muscular layer |
| ESF630 | SCC | F | 54 | NA | NA | Squamous cell carcinoma invade fibrosa. No |
| | | | | | | malignant cells adjacent to superior and |
| | | | | | | inferior stump. Regional LN: paraesphageal LN |
| | | | | | | (0/3), left stomach LN (0/2), hilum of lung LN |
| | | | | | | (0/3). IHC results: CD117 (+), Vim(+), Act(−), S- |
| | | | | | | 100(−). |
| ESM201 | SCC | M | 58 | T3N1M0 III | II-III | squamous cell carcinoma of middle thoracic |
| | | | | | | esophagus (medullary); Squamous cell |
| | | | | | | carcinoma from middle thoracic part of |
| | | | | | | esophagus, medullary type, grade II-III |
| | | | | | | (moderately-poorly differentiated), tumor |
| | | | | | | mass 4.5 cm × 1.5 cm × 1 cm, infiltrating into |
| | | | | | | fibrous membra |
| ESM026 | SCC | M | 62 | NA | I-II | multiple well-moderately differentiated |
| | | | | | | squamous cell carcinoma of esophagus; |
| | | | | | | Multiple squamous cell carcinoma from |
| | | | | | | esophagus, well-moderately differentiated, |
| | | | | | | carcinoma in situ, tumor mass 5 cm × 5 cm × 2 cm, |
| | | | | | | 2 cm × 2 cm × 1 cm, 1.5 cm × 1 cm × 1 cm. |
| ESF195 | SCC | F | 58 | T3N0M0, II A | II-III | Squamous cell carcinoma from middle |
| | | | | | | thoracic part of esophagus, uclerative type, |
| | | | | | | grade II-III (moderately and poorly |
| | | | | | | differentiated), tumor mass |
| | | | | | | 4 cm × 3.5 cm × 0.5 cm, infiltrating into fibrous |
| | | M | 53 | T2N0M0 II | II | moderately differentiated adenocarcinoma of |
| | | | | | | gastric cardia (ulcerative) |
| ESM178 | SCC | M | 57 | T3N1M0 III | II | squamous cell carcinoma of esophagus |
| | | | | | | (medullary); Squamous cell carcinoma from |
| | | | | | | middle thoracic part of esophagus, medullary |
| | | | | | | type, grade II (moderately differentiated), |
| | | | | | | tumor mass 3 cm × 2.5 cm × 1.8 cm, infiltrating |
| | | | | | | into fibrous membranes, no visible malignant c |
| ESF189 | SCC | F | 56 | T3N1M0 III | II | Squamous cell carcinoma from middle thoracic |
| | | | | | | part of esophagus, medullary type, grade II |
| | | | | | | (moderately differentiated), tumor mass |
| | | | | | | 4 cm × 3.5 cm × 1 cm, infiltrating into fibrous |
| | | | | | | membranes, no visible malignant cell |
| ESF191 | SCC | F | 59 | T3N1M0 III | II | squamous cell carcinoma of esophagus |
| | | | | | | (ulcerative); Squamous cell carcinoma from |
| | | | | | | middle thoracic part of esophagus, ulcerative |
| | | | | | | type, grade II (moderately differentiated), |
| | | | | | | tumor mass 5.5 cm × 4.5 cm × 0.8 cm, infiltrating |
| | | | | | | into fibrous membranes, no visible maligna |
| | | M | 68 | T3N0M0 II | II | moderately differentiated squamous cell |
| | | | | | | carcinoma of esophageal end part and gastric |
| | | | | | | cardia |

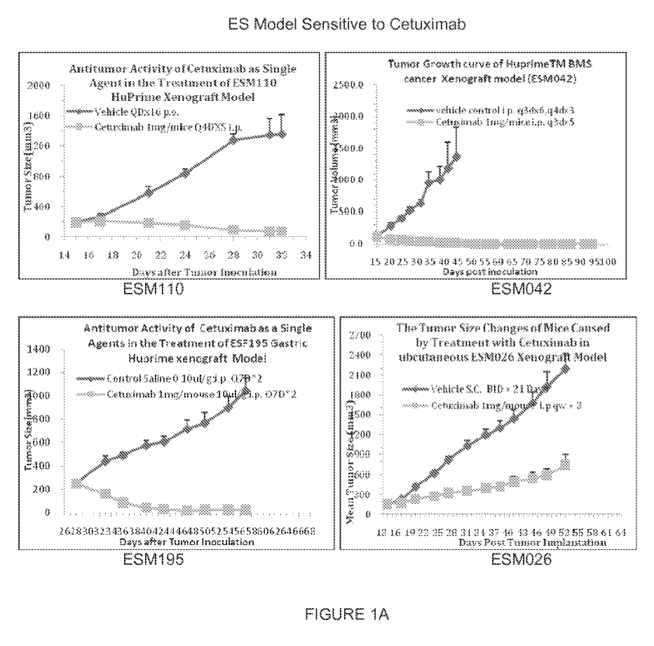

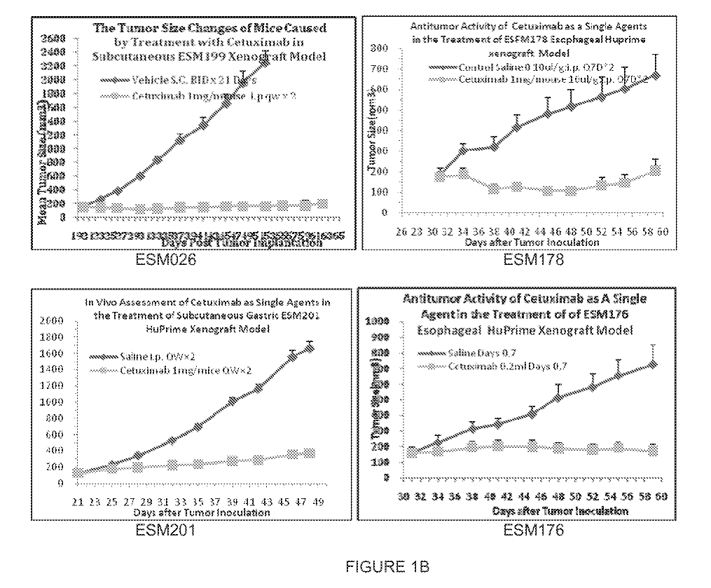

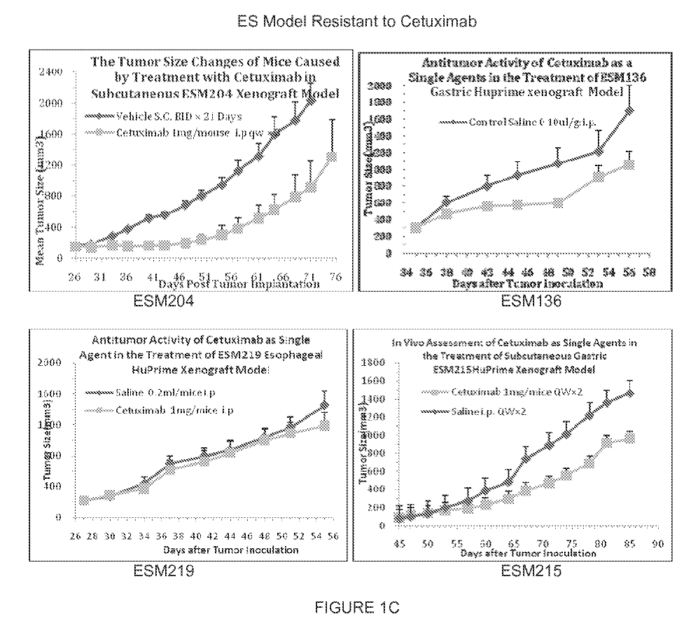

Second, these models were then subjected to once weekly cetuximab treatment for 2 weeks at 1 mg/mouse in order to assess their sensitivity to cetuximab. Interestingly, the treatment results demonstrated that a majority of ESC-SCC responded to cetuximab with high response rates (RR): “complete response” (CR, defined as ΔT/ΔC<0%) of 4/10, or 40%; partial response (PR, 0%<% ΔT/ΔC<50%) of 6/10; and no non-responder (NR, 50%<0% ΔT/ΔC) quantification of tumor response as measured by ΔT/ΔC values are summarized in Table 1. The representative antitumor activities of the models are also shown in FIG. 1.

This observation was surprising for several reasons. First, little clinical information was available on ESC-SCC response to cetuximab; and second, the exceptionally high RR were in sharp contrast to the significantly lower RR to cetuximab observed for other cancer types whose PDX models were assessed under the same treatment conditions, which include colorectal (CRC) (Chen D, et al. Cetuximab response in Asian CRC patient-derived xenografts is predicted by RAS pathway activation not KRAS mutation status. in submission), gastric carcinoma (GC) (Li et al., unpublished observations) and NSCLC (Yang M, et al. Squamous non-small cell lung cancer (NSCLC-SCC) patient-derived xenografts (PDX) from Asian patients have high response rate (RR) to cetuximab than those from non-SCC patients. in submission). The strong response to cetuximab by many of the ESC-SCC suggested that EGFR could play a significant role in driving their oncogenesis. Cetuximab thus could be an effective treatment for ESC-SCC, particularly for Asian patients, such as East Asian patients.

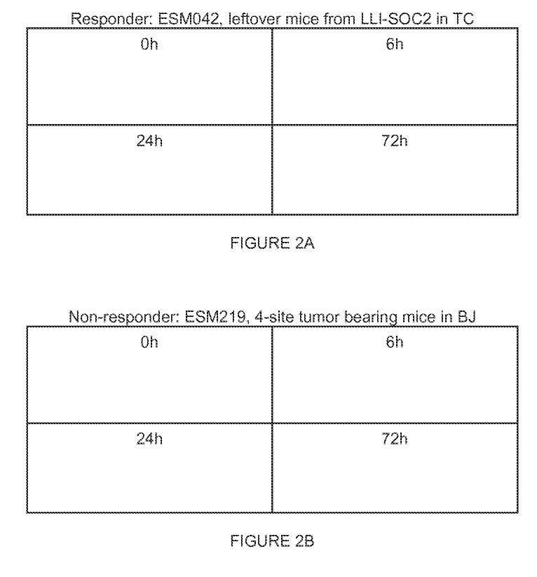

It was then investigated whether EGFR signaling was indeed inactivated in these tumors by cetuximab treatment by conducting a single dose pharmacodynamic study. The tumor bearing animals were treated with a single dose of cetuximab at 1 mg/kg and the tumors were harvested at different time points (2, 6 and 24 hours post dosing). The tissues were examined by IHC staining for pEGFR, along with pERK and pAKT, the two key downstream signaling biomarkers. The representative example of ES110 IHC analysis is shown in FIG. 2A (image) and 2B (score quantization). The results clearly demonstrated the reduction of all these signaling biomarkers.

EGFR gene amplification and/or over-expression seemed to be a strong oncogenic driver in a subset of ESC-SCC HuPrime® that showed the strongest response. Although nearly all the ESC-SCC models tested so far demonstrated responses, the degree of response varies greatly, and thus indicated genetic variations among the models may have dictated the response. Therefore genetic and genomic biomarkers that might have dictated the observed responses in these models were investigated. Since EGFR is the treatment target and apparent oncogenic driver for the responders, these models were focused on first for investigating EGFR.

EGFR expression was confirmed using IHC, a method commonly used in the clinic. The results demonstrated that all the ESC-SCC express EGFR at protein levels. Next, EGFR expression was investigated at the mRNA level using Affymetix HG-U219 GeneChip analysis. Interestingly, all of the degrees of the responses in all of these models correlated with the levels of EGFR (Table 1 and FIG. 3). This observation was plausible since the higher activity of EGFR via higher expression could drive the oncogenic transformation in these tumors and the inactivation by cetuximab could thus inhibit tumor growth.

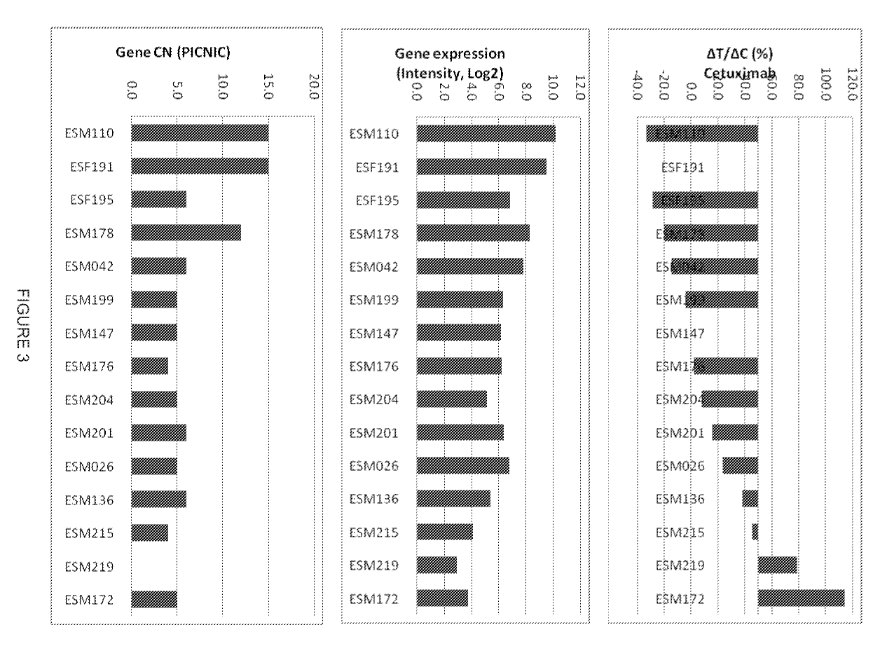

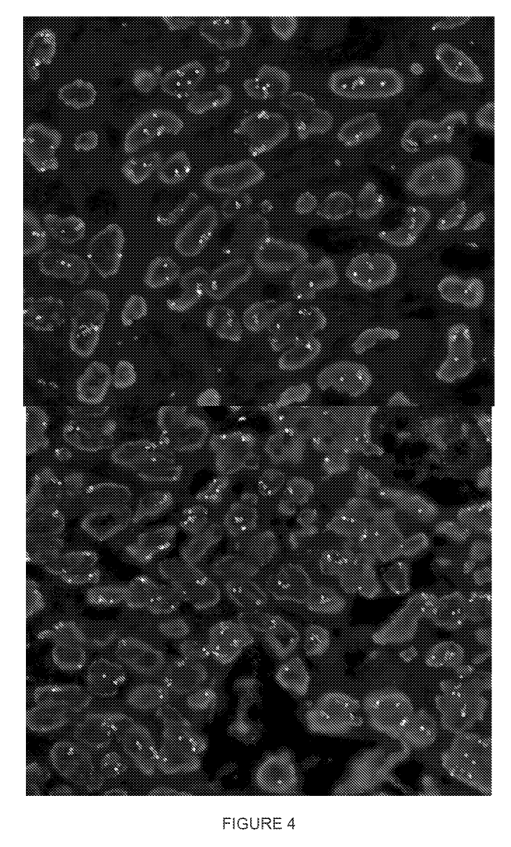

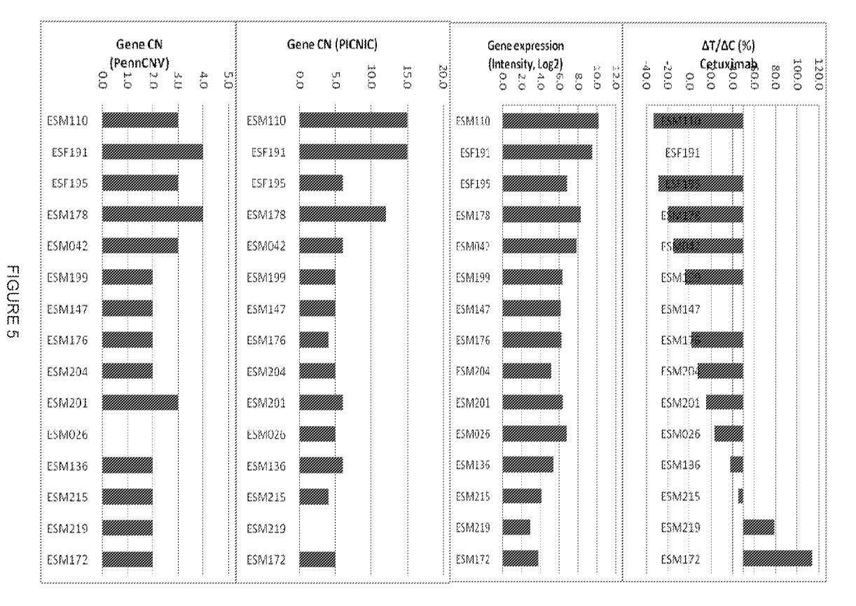

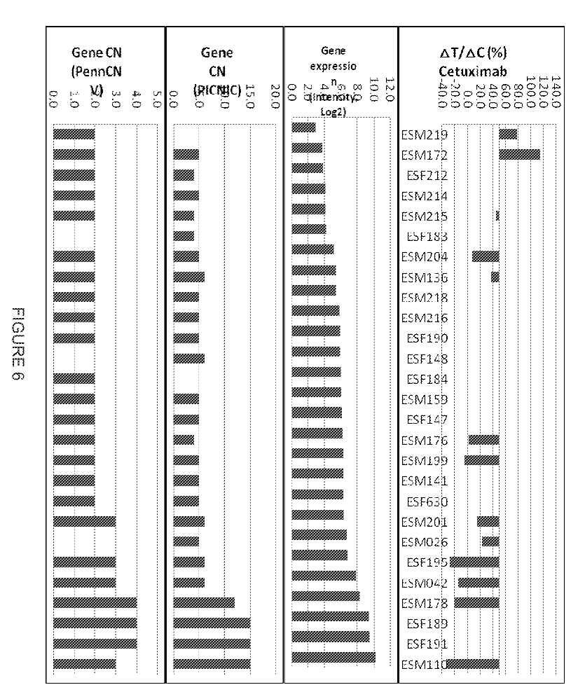

Furthermore, the genetic defects behind the higher EGFR expression were also investigated by first examining gene copy changes using Affymetrix SNP6 analysis and/or qPCR. Interestingly, all the responders/EGFR higher expressers have the corresponding EGFR gene amplification (Table 1 and FIG. 3). This observation suggested that EGFR gene amplification is likely the key oncogenic driver in the responders and a potential practical biomarker for predicting response to cetuximab in ESC-SCC. To further confirm the gene amplification, EGFR-fluorescence in situ hybridization, or FISH, a clinically practical assay, was also performed to assess EGFR gene amplification status of all these models. The FISH data indeed confirmed the observations seen by SNP6. FIG. 3, bottom panel, depicts GA110 FISH analysis as an example, clearly indicating EGFR amplification (FIG. 4, left panel). The clinically accepted FISH enables the development of companion diagnosis for cetuximab treatment in the clinic.

One of the important questions was whether the original tumors from which the PDXs were derived from also have the same gene amplifications, or higher expression, as seen in the models. To this end, the patient tumor samples were further analyzed with the samples tested. Among the tested patient samples, the amplifications were confirmed. FIG. 4, right panel, demonstrated that the original patient sample also has EGFR gene amplification.

Furthermore, several common oncogenes for activating mutations, including those in KRAS, EGFR, AKT, c-met, and BRAF, etc., were also analyzed. Interestingly, none of the tested models, regardless of the level of response to cetuximab, showed any activation mutations in the tested oncogenes, including KRAS, BRAF, AKT, PI3KC, c-met, c-kit, etc. (Table 1).

### DISCUSSION

This study clearly demonstrated that most ESC-SCC HuPrime® models established from Chinese patients respond to cetuximab to some degree. The extent of response correlated to the EGFR gene expression and copy numbers. Those with gene amplification and elevated expression had a significantly better response to cetuximab. This data therefore suggested that cetuximab could be a new effective treatment option for patients of ESC-SCC, particularly the patients from East Asia. In addition, the data also support that EGFR gene amplification as a predicative biomarker for the assumed responders to cetuximab in ESC-SCC patients. EGFR FISH assay for EGFR gene amplification are routinely used in the clinic and could be used as companion diagnostics for ESC-SCC treatment. Therefore, such a prospective trial can readily be implemented. Confirmation in clinic could facilitate regulatory approval of the use of cetuximab for treating ESC-SCC patients, offering the first target therapy for ESC-SCC patients.

The ESC-SCC HuPrime® model response to cetuximab was dependent on the high expression and high copy numbers of EGFR, in sharp contrast to NSCLC and CRC HuPrime® where the copy number and expression levels played significant lesser role (Chen D, et al. Cetuximab response in Asian CRC patient-derived xenografts is predicted by RAS pathway activation not KRAS mutation status, in submission and Yang M, et al. Squamous non-small cell lung cancer (NSCLC-SCC) patient-derived xenografts (PDX) from Asian patients have high response rate (RR) to cetuximab than those from non-SCC patients, in submission). The EGFR over-expressing (elevated gene copy number)-cetuximab response relationship in ESC-SCC was similar to the Her2 over-expressing (elevated gene copy number)-trastuzumab response relationship in GC. This similarity provides a clear feasible development and regulatory path for a companion diagnostic for cetuximab treatment of ESC-SCC, just as the companion diagnostic for trastuzumab.

In summary, among the 15 esophageal SCC PDX models tested, all responded to cetuximab to some degree, with a significantly higher response rate (RR) than those seen in other cancer types. The expression profiling and copy number variation analysis revealed that the response was directly related to EGFR gene amplification and high expression. This data suggests that EGFR is a key oncogenic driver for this disease. Cetuximab is a treatment option for esophageal SCC patients, and EGFR gene amplification/expression is a predictive biomarker for this subset of ESC.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions.

Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

1. A method for treating esophageal carcinoma in a patient comprising administering to the patient an effective amount of an anti-EGFR agent treatment.

2. The method of claim 1 wherein the patient has one or more EGFR biomarkers.

3. A method for treating esophageal carcinoma in a patient comprising detecting the presence or absence of one or more EGFR biomarkers and treating a patient with an anti-EGFR agent treatment when one or more EGFR biomarkers are present.

4. A method for identifying responder and/or nonresponder patients comprising detecting the presence or absence of one or more EGFR biomarkers in a biological sample from a patient with esophageal carcinoma, wherein the presence of one or more EGFR biomarkers is indicative of a responder to an anti-EGFR agent treatment and the absence of an EGFR biomarker is indicative of a nonresponder to an anti-EGFR agent treatment.

5. A method for determining a treatment regimen for treating esophageal carcinoma in a patient in need thereof comprising detecting the presence or absence of one or more EGFR biomarkers in a biological sample from a patient with esophageal carcinoma, wherein the presence of one or more EGFR biomarkers is indicative of a responder to an anti-EGFR agent treatment and the absence of an EGFR biomarker is indicative of a nonresponder to an anti-EGFR agent treatment, and treating said patient with an anti-EGFR agent treatment when one or more EGFR biomarkers are present.

6. A method for altering the treatment regimen of an anti-EGFR agent treatment comprising detecting the presence or absence of one or more EGFR biomarkers in a biological sample from a patient receiving an anti-EGFR agent treatment and altering the treatment regimen based on the presence of one or more EGFR biomarkers in said biological sample, wherein when one or more EGFR biomarkers are present the treatment is continued.

7. A method of selecting a patient with esophageal carcinoma for treatment with an anti-EGFR agent treatment comprising detecting the presence or absence of one or more EGFR biomarkers in a biological sample from said patient, wherein the presence of one or more EGFR biomarkers is indicative of a responder to an anti-EGFR agent treatment and the absence of an EGFR biomarker is indicative of a nonresponder to an anti-EGFR agent treatment, and selecting for treatment with an anti-EGFR agent treatment those patients with the presence of one or more EGFR biomarkers.

8. The method of any of claims 2-7 wherein the EGFR biomarker is selected from EGFR gene amplification, EGFR expression, EGFR RNA levels, constitutively active EGFR, EGFR activity, EGFR pathway activation or EGFR pathway signaling.

9. The method of claim 8 wherein said EGFR gene amplification is an increase in the EGFR gene copy number compared to a predetermined reference EGFR gene copy number.

10. The method of claim 9 wherein the EGFR gene copy number is selected from at least 3, 4, 5 and 6 copies or more.

11. The method of any of claims 1-11 wherein the patient is of Asian descent.

12. The method of any of claims 1-11 wherein the esophageal carcinoma is esophageal squamous cell carcinoma.

13. The method of any of claims 1-12 wherein the anti-EGFR agent treatment is an anti-EGFR antibody treatment.

14. The method of any of claims 1-12 wherein the anti-EGFR agent treatment is a treatment of cetuximab, a biosimilar or a derivative thereof.

15. The method of any of claims 1-12 wherein the anti-EGFR agent treatment is co-administered with one or more chemotherapeutics, radiation therapeutics chemoradiation therapeutics or targeted therapeutics.

16. The method of claim 15 wherein said chemotherapeutic is selected from vinblastine, vincristine, dactinomycin, daunorubicin, doxorubicin, etoposide, mithramycin, paclitaxel, docetaxel, cisplatin, carboplatin, fluorouracil, folinic acid and irinotecan.

17. The method of claim 15 wherein the targeted therapeutic is selected from bevacizumab, trastuzumab, erlotinib, panitumumab, sorafenib, infliximab, adalimumab, basiliximab, daclizumab and omalizumab.

18. A method of providing useful information for determining, evaluating or monitoring the treatment or efficacy of treatment of esophageal carcinoma with an anti-EGFR antibody treatment comprising determining the presence or absence of one or more EGFR biomarkers in a biological sample from a patient and providing the determination of the presence or absence of said one or more EGFR biomarkers to an entity that provides a determination or evaluation of the treatment or efficacy based on the presence or absence of one or more EGFR biomarkers.

19. A kit comprising a reagent for measuring one or more EGFR biomarkers in a biological sample and optionally an instruction for using the measurement, wherein the presence of one or more EGFR biomarkers is indicative of a responder to an anti-EGFR agent treatment and the absence of one or more EGFR biomarkers is indicative of a nonresponder to an anti-EGFR agent treatment.

20. The method of claim 7, wherein the method comprises detecting the protein expression level of EGFR.

21. The method of claim 20, wherein the protein expression level is determined by immunohistochemistry (IHC), western blot, protein immunestaining, protein immuneprecipitation, immuneeletrophoresis, immunoblotting, BCA assay, spectrophotometry, mass spectrometry or enzyme assay.


SEQUENCE LISTING (40 sequences)

SEQ ID NO: 1 - DNA, 21 bp, Artificial Sequence
  EGFR-EXON19-F
gtgcatcgctggtaacatcca

SEQ ID NO: 2 - DNA, 23 bp, Artificial Sequence
  EGFR-EXON19-R
ggagatgagcagggtctagagca

SEQ ID NO: 3 - DNA, 20 bp, Artificial Sequence
  EGFR-EXON20-F
cgcattcatgcgtcttcacc

SEQ ID NO: 4 - DNA, 20 bp, Artificial Sequence
  EGFR-EXON20-R
ctatcccaggagcgcagacc

SEQ ID NO: 5 - DNA, 21 bp, Artificial Sequence
  EGFR-EXON21-F
tggcatgaacatgaccctgaa

SEQ ID NO: 6 - DNA, 19 bp, Artificial Sequence
  EGFR-EXON21-R
cagcctggtccctggtgtc

SEQ ID NO: 7 - DNA, 21 bp, Artificial Sequence
  KRAS-EXON2-F
ttatgtgtgacatgttctaat

SEQ ID NO: 8 - DNA, 21 bp, Artificial Sequence
  KRAS-EXON2-R
agaatggtcctgcaccagtaa

SEQ ID NO: 9 - DNA, 20 bp, Artificial Sequence
  KRAS-EXON3-F
tcaagtcctttgcccatttt

SEQ ID NO: 10 - DNA, 20 bp, Artificial Sequence
  KRAS-EXON3-R
tgcatggcattagcaaagac

SEQ ID NO: 11 - DNA, 21 bp, Artificial Sequence
  KRAS-EXON4-F
ttgtggacaggttttgaaaga

SEQ ID NO: 12 - DNA, 20 bp, Artificial Sequence
  KRAS-EXON4-R
agaagcaatgccctctcaag

SEQ ID NO: 13 - DNA, 19 bp, Artificial Sequence
  PI3K-Exon 1, F
ctccacgaccatcatcagg

SEQ ID NO: 14 - DNA, 26 bp, Artificial Sequence
  PI3K-Exon 1, R
gattacgaaggtattggtttagacag

SEQ ID NO: 15 - DNA, 22 bp, Artificial Sequence
  PI3K-Exon 9, F
gattggttctttcctgtctctg

SEQ ID NO: 16 - DNA, 26 bp, Artificial Sequence
  PI3K-Exon 9, R
ccacaaatatcaatttacaaccattg

SEQ ID NO: 17 - DNA, 21 bp, Artificial Sequence
  PI3K-Exon 20 F
tggggtaaagggaatcaaaag

SEQ ID NO: 18 - DNA, 20 bp, Artificial Sequence
  PI3K-Exon 20 R
cctatgcaatcggtctttgc

SEQ ID NO: 19 - DNA, 21 bp, Artificial Sequence
  c-MET-Exon 14, F
tgggcactgggtcaaagtctc

SEQ ID NO: 20 - DNA, 25 bp, Artificial Sequence
  c-MET-Exon 14, R
aacaatgtcacaacccactgaggta

SEQ ID NO: 21 - DNA, 23 bp, Artificial Sequence
  c-MET-Exon 16, F
attaaatgttacgcagtgctaac

SEQ ID NO: 22 - DNA, 21 bp, Artificial Sequence
  c-MET-Exon 16, R
ggttgcaaaccacaaaagtat

SEQ ID NO: 23 - DNA, 24 bp, Artificial Sequence
  c-MET-Exon 17, F
gtattcactgttccataatgaagt

SEQ ID NO: 24 - DNA, 22 bp, Artificial Sequence
  c-MET-Exon 17, R
gatggctggcttacagctagtt

SEQ ID NO: 25 - DNA, 24 bp, Artificial Sequence
  c-MET-Exon 18, F
aacagtagatgcttagtttatgct

SEQ ID NO: 26 - DNA, 22 bp, Artificial Sequence
  c-MET-Exon 18, R
aacagattcctccttgtcactt

SEQ ID NO: 27 - DNA, 22 bp, Artificial Sequence
  c-MET-Exon 19, F
ttctatttcagccacgggtaat

SEQ ID NO: 28 - DNA, 24 bp, Artificial Sequence
  c-MET-Exon 19, R
atgaaagtaaaagaggagaaactc

SEQ ID NO: 29 - DNA, 20 bp, Artificial Sequence
  c-MET-Exon 21, F
caccctaaagccgaaatgcg

SEQ ID NO: 30 - DNA, 20 bp, Artificial Sequence
  c-MET-Exon 21, R
caccctaaagccgaaatgcg

SEQ ID NO: 31 - DNA, 18 bp, Artificial Sequence
  AKT-Exon 3, F
acatctgtcctggcacac

SEQ ID NO: 32 - DNA, 18 bp, Artificial Sequence
  AKT-Exon 3, R
gccagtgcttgttgcttg

SEQ ID NO: 33 - DNA, 19 bp, Artificial Sequence
  BRAF-Exon 15, F
ctcttcataatgcttgctc

SEQ ID NO: 34 - DNA, 19 bp, Artificial Sequence
  BRAF-Exon 15, R
gtgaatactgggaactatg

SEQ ID NO: 35 - DNA, 20 bp, Artificial Sequence
  ERK-Exon 2, F
actttaccaacttgccttct

SEQ ID NO: 36 - DNA, 18 bp, Artificial Sequence
  ERK-Exon 2, R
tcacaacaaaccatccct

SEQ ID NO: 37 - DNA, 15 bp, Artificial Sequence
  ERK-Exon 8, F
tgccttacccataac

SEQ ID NO: 38 - DNA, 19 bp, Artificial Sequence
  ERK-Exon 8, R
ggaccttgaggaacataat

SEQ ID NO: 39 - DNA, 20 bp, Artificial Sequence
  EGFR-F
catggtgagggctgaggtga

SEQ ID NO: 40 - DNA, 20 bp, Artificial Sequence
  EGFR-R
ccccaccagaccatgagagg